(19) United States
(12) Patent Application Publication
(10) Pub. No.: US 2002/0062114 A1
(43) Pub. Date: May 23, 2002

(54) Absorbent articles

(76) Inventors: Kouichi Murai, Tokyo (JP); Yuria Murai, Tokyo (JP)

Correspondence Address: Liniak, Berenato, Longacre & White, Ste. 240, 6550 Rock Spring Drive, Bethesda, MD, 20817, US

(21) Appl. No.: 09/988,822

(22) Filed: Nov. 20, 2001

(30) Foreign Application Priority Data
Nov. 21, 2000 (JP) 2000-353668

(51) Int. Cl. A61F 13/15; G01N 5/02

(52) U.S. Cl. 604/385.01; 73/73; 73/74

(57) ABSTRACT

An absorbent article that make it possible to assess the amount of the liquid discharging from human body and other spots as well as spreading on a floor without depending on determining a weight difference of the absorbent body (weight after absorption-weight before absorption). An absorbent article comprises a liquid pervious topsheet 101, a liquid impervious backsheet 102 and absorbent body 103 having the capability of retaining liquids positioned therebetween, the absorbent article comprising a topsheet having a mesh-type scale 104 on its outer surface.

### BACKGROUND OF THE INVENTION

1. Field of the Invention

The present invention relates to absorbent articles having the capability of easily absorbing blood, bodily exudates and other aqueous fluids (Hereinafter, the term “liquid” is used to mean the blood, bodily exudates and other aqueous fluids, unless otherwise indicated) by assessing an absorbed amount thereof. More particularly, the present invention relates to an absorbent article appropriately applicable to the articles such as a sanitary napkin, disposable diaper, disposable pant diaper (sheet-form absorbent articles) and a tampon and the like.

2. Description of the Related Art

Currently, difference in the weight of absorbent body (napkin, tampon and the like) between before and after its application is determined to assess the amount of bleeding, for example, during menstruation and hemorrhage from wound. This procedure is likely associated with disadvantages of erroneous assessment due to possible evaporation of water from the blood. Furthermore, this procedure is inevitably associated with troublesome manipulations to assess the weight, and, thus, a need exists for development of more simple and immediate manipulation to assess the amount of bleeding.

In addition to assessing the amount of bleeding from human body, a great need exists for simple procedures to assess the trace amount of the liquids discharging through a tiny space in a joining part of liquid-transporting pipeline, the amount of the liquid spreading on a floor and the like.

### SUMMARY OF THE INVENTION

It is an object of the present invention is to provide absorbent articles that make it possible to assess the amount of the liquid, for example, discharging from human body and other spots or spreading on a floor without depending on determining a weight difference of the absorbent body (weight after absorption-weight before absorption).

The present invention provides an absorbent article comprising a liquid pervious topsheet, a liquid impervious backsheet and absorbent body having the capability of retaining liquids positioned therebetween, and, particularly, the absorbent article comprising a topsheet having a mesh-type scale on its outer surface.

Absorbent articles include a sanitary napkin, disposable diaper, disposable pant diaper (sheet-form absorbent articles) and the like.

Furthermore, the present invention provides the absorbent article comprising an absorbent body having its thickness kept constant evenly throughout the entire body.

The structure makes it possible to easily assess the amount of the liquid absorbed into the absorbent body by multiplying the per-unit area maximal absorbent capacity of the absorbent body by an area of liquid-absorption mark remaining on the surface of absorbent body.

In addition, the present invention provides the absorbent article comprising an absorbent body impregnated with coloring agents that develop a color upon contacting with moisture. Even when the liquids are clear, the maneuver makes it possible to easily measure an area of liquid-absorption mark and, thus, to assess the amount of the liquid absorbed into the absorbent article.

Furthermore, the present invention provides the absorbent article comprising a substantially cylinder-shape absorbent body and a pair of removal cords firmly attached to the absorbent body, the absorbent article comprising the absorbent body having a scale arranged along longitudinal axis on its side surface for assessing the amount of the absorbed liquid.

### BRIEF DESCRIPTION OF THE DRAWINGS

FIG. 1 is a perspective view of the sheet-form absorbent article according to the present invention.

FIG. 2 is a perspective view of the napkin according to the present invention.

FIG. 3 is a cross sectional view taken along line 3-3 of FIG. 2,

FIG. 4 is a perspective view of the tampon according top the present invention.

### DETAILED DESCRIPTION OF THE PREFERRED EMBODIMENT

The absorbent articles according to the present invention can be of any shapes including not only sheet-form absorbent articles such as napkin, disposable diaper and the like but also disposable pant diaper incorporating a sheet-form absorbent article as its portion.

The absorbent articles are used under close contact with the liquids and the spot where the liquids are discharging (for example, wound, female genital organ, tiny pore of the liquid-transporting pipeline where liquids are discharging, the liquids spreading on a floor and the like). Owing to a liquid pervious nature of the topsheet, the liquids can be absorbed into the absorbent body having the capability of retaining liquids by penetration through the topsheet. Furthermore, owing to a liquid impervious nature of the backsheet, the liquids having been absorbed into the absorbent body can be retained in absorbent articles, escaping from leakage outside.

Topsheet, backsheet and absorbent body can be made of any materials well known in the art for use in disposable diaper, sanitary napkin and the like. The topsheet is made of, for example, hydrophilic or hydrophobic unwoven webs, porous formed plastic film or laminate of their layers. The absorbent body is made of, for example, a material consisting of mainly flap pulp in combination with high-absorbent polymer. The backsheet is made of, for example, breathable plastic film or laminate of this with hydrophobic unwoven webs. A mesh-type scale is laid out on the outer surface of the topsheet by means well known in the art including, for example, printing and embossing finish.

The absorbent article of the invention having a mesh-type scale laid out on the outer surface of the topsheet makes it possible to assess the amount (in either mass or volume) of the liquid absorbed into the absorbent article by counting the number of mesh openings covering the liquid-absorption mark (absorption mark of blood and the like) (i.e. by measuring an area of the liquid-absorption mark).

The absorbent body of the absorbent article of the invention formed such that thickness of the absorbent body is kept constant evenly throughout the entire body makes it possible to assess the amount (in either mass or volume) of the liquid absorbed into the absorbent article by multiplying the number of mesh openings covering the liquid-absorption mark (absorption mark of blood and the like) by per-mesh opening maximal absorbent capacity. In order for this assessment to be feasible, it needs to know in advance the per-mesh opening maximal absorbent capacity by preliminary testing.

Constant thickness of the absorbent body may be difficult to be kept evenly throughout the entire body when the absorbent article is applied in the form of napkin because of an expectation for closer contact with wearer's body. In this case, however, count of the number of mesh openings covering the liquid-absorption mark (absorption mark of blood and the like) makes it possible to give an accurate assessment of the amount (either in mass or volume) of the absorbed liquid if the size of each mesh opening is varied such that the volume of the absorbent body beneath each mesh becomes equal. More particularly, a mesh-type scale is printed or laid out on the topsheet such that a mesh opening is smaller corresponding to a more thick portion of the absorbent body and a mesh opening is larger corresponding to a less thick portion of the absorbent body.

Such being the case, it needs to know in advance the per-mesh opening maximal absorbent capacity by preliminary testing for the absorbent body of uneven thickness.

Layout of the mesh-type scale on the surface of the topsheet is not necessarily subjected to restriction in terms of a longer side/shorter side ratio and of a size of mesh opening provided that each mesh opening is of rectangular or square shape arranged by an area ratio such that the volume of a portion of the absorbent body directly beneath each mesh opening becomes equal.

Smaller mesh opening is generally preferable to make an assessment of the amount of the absorbed liquid with higher precision. On the other hands, larger mesh opening is more suitable to the purpose of counting the number of mesh openings covering the liquid-absorption mark with ease. Furthermore, larger mesh opening is suitable to assess a larger sized mark remaining following absorption of larger amount of the liquid.

In embodiments of the present investigation, the size of mesh opening is preferably determined taking account of balanced arrangement of the factors. For example, when the absorbent article of the invention is applied to assessment of the amount of the menstrual blood in the form of a napkin, a scale is preferably laid out to form a mesh selected from those having a mesh opening of about 5 to 20 mm width depending upon a needed precision in assessing the amount of the absorbed liquid.

Accompanying graduation figure along with each graduation line as an aid in reading the number of mesh openings covering the liquid-absorption mark makes it possible to ascertain the number of mesh openings covering the liquid-absorption mark with ease.

The absorbent body is not necessarily subjected to restriction in terms of its thickness, and, thus, it is preferable to use the absorbent body of any thickness selected from less (about several millimeters) to more (about several centimeters) thick ones depending upon the amount of liquid discharges to be assessed.

The sheet-form absorbent article is not necessarily subjected to restriction in terms of its size and shape provided that the mesh-type scale is laid out on its topsheet.

Coloring agents that develop a color upon contacting with moisture impregnated in the absorbent body makes it possible to assess the amount of the discharging liquid even if the discharge is of clear liquid. Silica-gel powder and the like can be used as the coloring agents.

In the following, the absorbent articles of the present invention will be described in greater detail with reference to embodiment shown in the accompanying drawings.

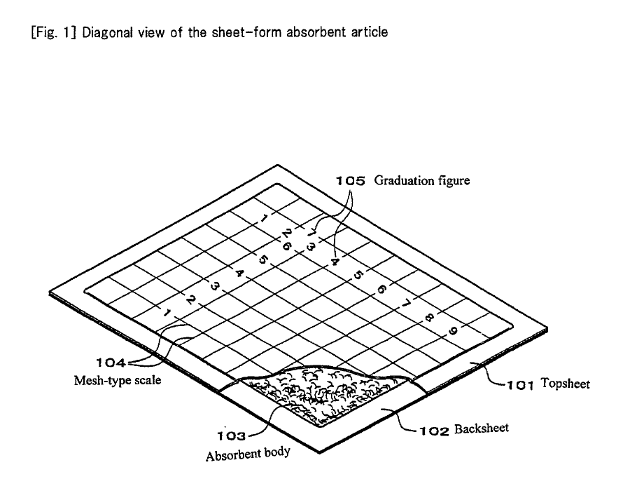

FIG. 1 is a diagonal view of the sheet-form absorbent article of the present invention from a direction of the topsheet side.

As shown in FIG. 1, the rectangle-shape absorbent article comprises of a liquid pervious topsheet 101 that comes into contact with the liquid to be absorbed, a liquid impervious backsheet 102 that is arranged so as to form the back side of the absorbent article and an absorbent body 103 capable of retaining the liquid that is arranged so as to be sandwiched therebetween. In a preferred embodiment of the absorbent article as shown in FIG. 1, the absorbent body 103 is kept constant in terms of its thickness evenly throughout the entire body. The topsheet 101 has a scale 104 comprising of graduation lines arranged at constant intervals so as to form a series of mesh opening of square shape each (designated as “mesh-type scale”, hereinafter) printed on its outer surface. Furthermore, each graduation line of the scale 104 is accompanied by each graduation figure 105 as an aid. These figures are not needed to agree with actual length or mass because of their roles only as a guide in ascertaining the number of mesh openings covering the liquid-absorption mark.

Examples of use of the absorbent sheet shown in FIG. 1 include (1) Postoperative use in assessing the blood amount exuding from the site of suture closure by applying the sheet-form absorbent article of an appropriate size such that the absorbent article comes into close contact with the site of suture closure at its topsheet side and (2) Use in exact assessment of the amount of the liquid spreading on a floor by absorbing the liquid spreading on a floor into the sheet-form absorbent article of the present invention through its close contact with the liquid concerned.

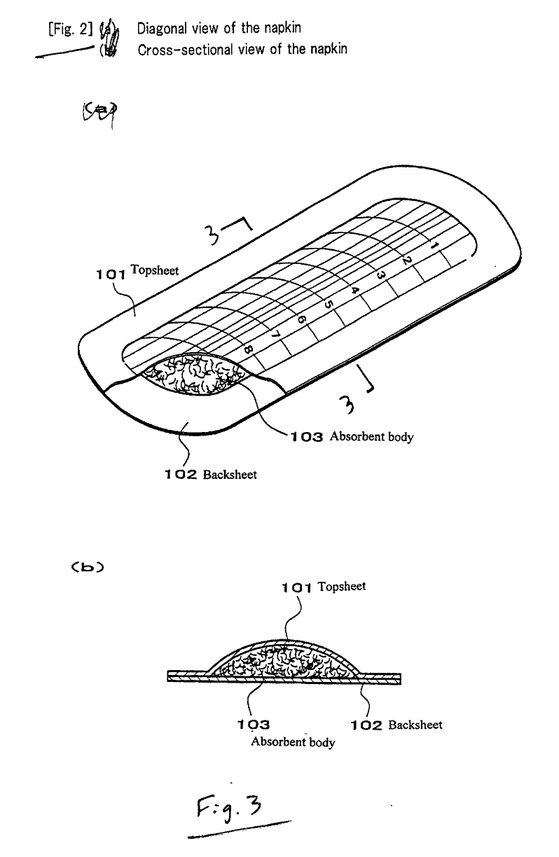

FIG. 2(a) is a diagonal view of another embodiment of the absorbent article of the present invention (napkin) and FIG. 2(b) is a cross sectional view of the napkin shown in FIG. 2(a).

The napkin is formed in substantially rectangular shape with 4 edges trimmed round so as to adapt to face the body of the wearer. Description is omitted on the portions common to those shown in FIG. 1 except otherwise indication by a corresponding number.

The napkin of an embodiment of the absorbent article of the present invention incorporates the absorbent body of gently upward convexed shape as represented by a cross-sectional view so as to adapt to face closely the body of the wearer (FIG. 2(b)). Accordingly, opening width of the mesh-type scale 104 on the napkin is adjusted such that the volume of the absorbent body beneath each mesh opening of the mesh-type scale becomes equal. More particularly, mesh openings are smallest in the middle portion of the absorbent body where its thickness is largest and mesh openings become larger along the direction toward its periphery.

Using the napkin shown as an embodiment of the present invention makes it easier to assess the amount of the blood discharged as a result of counting the number of mesh openings covering the blood-absorption mark.

The absorbent article according to claim 4 of the invention is preferably used in the form of a tampon. The absorbent body is principally formed in a substantially cylindrical shape, and, however, taking account of its use as an insert in the vaginal cavity, the most suitable form is of a cylinder with its edges trimmed off and its distal end (insertion end) formed in hemispheric shape. Any materials well known in the art can be used also as the materials of the absorbent body of the present invention. Taking hydrophilic fibers such as absorbent cotton, rayon and the like as an example, the tampon of an embodiment of the present invention is manufactured by the following procedures: firstly, the fibers are separated from each other and, then, thus separated fibers are mixed again to prepare a belt-form absorbent mass called fleece (cotton mass); thus prepared fleece is cut into fragments and a pair of removal cords is attached to the end of each of the fragments; the fragments are treated to form a cylinder-shape roll and thus formed cylinder-shape roll is finally subjected to heat compression.

The absorbent body has a scale arranged along longitudinal axis on its side surface for assessing the amount of the menstrual blood. The scale may be formed in any style, and, however, preferred style includes, for example, a scale comprising of a series of graduation lines arranged along longitudinal axis and at such intervals that the fractional volume of the absorbent body each cross-sectioned at each of the graduation lines becomes equal or a mesh-type scale laid out on its side surface such that a series of rectangular sections are formed.

The absorbent article according to claim 4 of the present invention (tampon) absorbs menstrual blood when inserted into the vaginal cavity during which the blood-absorption mark extends covering more and more graduation lines in a direction from the distal end toward the proximal end, and, thus, it is possible to assess the amount of menstrual bleeding by ascertaining the graduation line reached by a front of the blood-absorption mark (an area of the blood-absorption mark).

The tampon is different in diameter and blood absorbent capacity of its absorbent body depending upon its type and receives pressure during its use in the vaginal cavity, and, thus, it is preferable to understand in advance the relationship between the amount of the blood absorbed into the tampon (either in volume or mass) and the graduation line to be reached by a front of the blood-absorption mark (an area of the blood-absorption mark) under pressure corresponding to the intravaginal pressure.

Owing to this maneuver, the absorbent article according to claim 4 of the present invention (tampon) makes it easier to assess the amount of menstrual bleeding by ascertaining the graduation line reached by a front of the blood-absorption mark (an area of the blood-absorption mark).

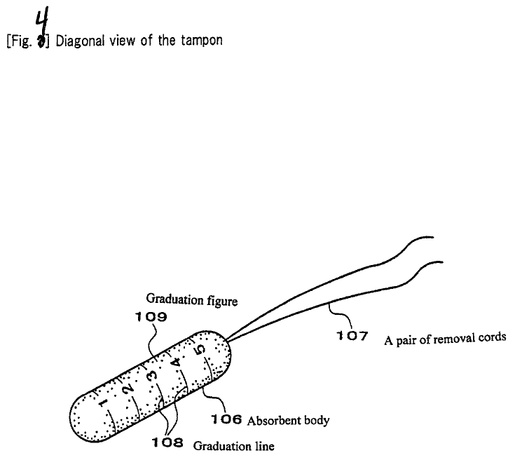

FIG. 3 shows an embodiment of the absorbent article according to claim 4 of the present invention (tampon). The absorbent body 106 is formed in substantially cylindrical shape with the distal (insertion) end and proximal end formed in hemispherical shape, and a pair of removal cords 107 is attached to the proximal end. Circle-form graduation lines 108 are printed on the outer surface of the absorbent body. The tampon of an embodiment of the present invention has graduation figures 109 printed as an aid in reading the amount of the absorbed blood. Using the tampon of an embodiment of the present invention makes it possible to assess the amount of bleeding with ease by ascertaining the graduation line reached by a front of the blood-absorption mark.

The advantage of the present invention comprises in particular in the fact that the absorbent article of the present invention provides an easier means of assessment of the amount of the liquid absorbed into the absorbent body replacing the conventional means wherein the weight is measured.

What is claimed is:

1. An absorbent article comprising: a liquid pervious topsheet, a liquid impervious backsheet; and an absorbent body having a capability of retaining liquids positioned between the topsheet and the backsheet, the topsheet having a mesh-type scale on its outer surface.

2. The absorbent article according to claim 1, wherein a thickness of the absorbent body is kept constant evenly throughout the entire absorbent body.

3. The absorbent article according to claim 1, wherein the absorbent body is impregnated with coloring agents that develop a color upon contact with moisture.

4. An absorbent article comprising a substantially cylinder-shape absorbent body and a pair of removal cords firmly attached to the absorbent body, the absorbent body having a scale arranged along a longitudinal axis on a side surface thereof for assessing an amount of absorbed liquid.

5. The absorbent article according to claim 2, wherein the absorbent body is impregnated with coloring agents that develop a color upon contacting with moisture.
